(19) United States
(12) Patent Application Publication
(10) Pub. No.: US 2006/0258685 A1
(43) Pub. Date: Nov. 16, 2006

(54) Substituted pyrazolopyrimidines, methods for the production thereof, use of the same for controlling pathogenic fungi, and agents containing said compounds

(76) Inventors: Olivier Wagner, Neustadt (DE); Thomas Grote, Wachenheim (DE); Carsten Blettner, Repulse Bay Road (DE); Markus Gewehr, Kastellaun (DE); Wassilios Grammenos, Ludwigshafen (DE); Andreas Gypser, Mannheim (DE); Bernd Muller, Frankenthal (DE); Joachim Rheinheimer, Ludwigshafen (DE); Peter Schafer, Ottersheim (DE); Frank Schieweck, Hessheim (DE); Anja Schwogler, Mannheim (DE); Jordi Tormo i Blasco, Weinheim (DE); Alan Akers, Avenue de Strasbourg (DE); John-Bryan Speakman, Bobenheim (DE); Michael Rack, Heidelberg (DE); Reinhard Stierl, Freinsheim (DE); Maria Scherer, Godramstein (DE); Ulrich Schofl, Bruhl (DE); Siegfried Strathmann, Limburgerhof (DE)

Correspondence Address: BIRCH STEWART KOLASCH & BIRCH, PO BOX 747, FALLS CHURCH, VA, 22040-0747, US

(21) Appl. No.: 10/558,251

(22) PCT Filed: May 27, 2004
(86) PCT No.: PCT/EP04/05694

§ 371 Date: Nov. 21, 2005

(30) Foreign Application Priority Data
Jun. 3, 2003 (DE) 103252444
Aug. 4, 2003 (DE) 103359605
Feb. 5, 2004 (DE) 1020040059101

(51) Int. Cl. A61K 31/519; C07D 487/04

(52) U.S. Cl. 514/259.3; 544/281

(57) ABSTRACT

The invention relates to substituted pyrazolopyrimidines of formula (I) wherein the substituents have the following designations: L represents halogen, alkyl, halogenalkyl, alkenyl, alkoxy, amino, NHR, NR₂, cyano, S(═O)ₙA¹ or C(═O)A², R representing alkyl or alkylcarbonyl, A¹ representing hydrogen, hydroxy, alkyl, alkylamino or dialkylamino, n representing 0, 1 or 2, and A² representing alkenyl, alkoxy, halogenalkoxy or one of the groups cited for A¹; m represents 0 or 1 to 5; R¹ represents alkyl, halogenalkyl, cycloalkyl, halogencycloalkyl, alkenyl, alkadienyl, halogenalkenyl, cycloalkenyl, alkinyl, halogenalkinyl or cycloalkinyl, phenyl, naphthyl, or a five-membered to ten-membered saturated, partially unsaturated or aromatic heterocycle containing between one and four heteroatoms from the group O, N or S; and R² represents hydrogen or one of the groups cited for R¹. Together with the nitrogen atom to which they are bonded, R¹ and R² can form a five-membered to six-membered ring that can be interrupted by an atom from the groups O, N and S, and R¹ and/or R² can also be substituted according to the description. Furthermore, in formula (I): X represents halogen, cyano, OH, alkyl, alkoxy or halogenalkoxy; Y represents a five-membered to ten-membered saturated, partially unsaturated or aromatic heterocycle according to the description, or a group X or another group according to the description; p represents 1 or 2, the groups Y being potentially different when p=2; and p represents 0, when X is according to the description. The invention also relates to methods and intermediate products for producing said compounds, agents containing the same, and the use thereof for controlling phytopathogenic fungi.

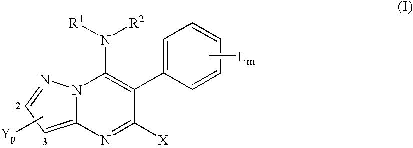

The present invention relates to substituted pyrazolopyrimidines of the formula I

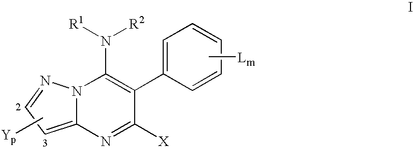

in which the substituents are as defined below:

L independently of one another are halogen, C₁-C₆-alkyl, C₂-C₆-alkenyl, C₁-C₆-haloalkyl, C₁-C₆-alkoxy, amino, NHR, NR₂, cyano, S(═O)ₙA¹ or C(═O)A²,

R is C₁-C₈-alkyl or C₁-C₈-alkylcarbonyl;

A¹ is hydrogen, hydroxyl, C₁-C₈-alkyl, C₁-C₈-alkylamino or di-(C₁-C₈-alkyl)amino;

n is 0, 1 or 2;

A² is C₂-C₈-alkenyl, C₁-C₈-alkoxy, C₁-C₆-haloalkoxy or one of the groups mentioned under A¹;

m is 0 or 1, 2, 3, 4 or 5;

R¹ is C₁-C₈-alkyl, C₁-C₈-haloalkyl, C₃-C₆-cycloalkyl, C₃-C₆-halocycloalkyl, C₂-C₈-alkenyl, C₄-C₁₀-alkadienyl, C₂-C₈-haloalkenyl, C₃-C₆-cycloalkenyl, C₂-C₈-alkynyl, C₂-C₈-haloalkynyl or C₃-C₆-cycloalkynyl, phenyl, naphthyl or a five- to ten-membered saturated, partially unsaturated or aromatic heterocycle which contains one to four heteroatoms from the group consisting of O, N and S;

R² is hydrogen or one of the groups mentioned under R¹;

R¹ and R² together with the nitrogen atom to which they are attached may also form a five- or six-membered ring which may be interrupted by an atom from the group consisting of O, N and S and/or which may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain;

where R¹ and/or R² may be substituted by one to four identical or different groups R^{a}:

R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, or a five- to ten-membered saturated, partially unsaturated or aromatic heterocycle which contains one to four heteroatoms from the group consisting of O, N and S,

where these aliphatic, alicyclic or aromatic groups for their part may be partially or fully halogenated or may carry one to three groups R^{b}:

R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups mentioned in these radicals contain 2 to 8 carbon atoms;

and/or one to three of the following radicals:

cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;

X is halogen, cyano, OH, C₁-C₄-alkyl, C₁-C₄-alkoxy or C₁-C₂-haloalkoxy,

Y is a five- to ten-membered saturated, partially unsaturated or aromatic heterocycle which contains one to four heteratoms from the group consisting of O, N and S, or one of the groups mentioned under X (which groups may be substituted by one to four identical or different groups R^{a}), nitro, amino, —CHO, —NHCO—NH—C₁-C₆-alkyl, —NHCO—O—C₁-C₆-alkyl, —CO—NH₂;

p is 1 or 2, where the groups Y may be different if p=2;

p is O if the group X is cyano, C₁-C₄-alkyl, C₁-C₄-alkoxy or C₁-C₄-haloalkoxy.

Moreover, the invention relates to processes and intermediates for preparing these compounds, to compositions comprising them and to their use for controlling phytopathogenic harmful fungi.

Pyrazolopyrimidines are known in a general manner from U.S. Pat. No. 4,567,263, WO 96/35690 and U.S. Pat. No. 5,817,663. WO 02/48151 discloses 6-phenylpyrazolopyrimidines in which the phenyl group is substituted by one to four groups. EP-A 71 792 describes 7-amino-pyrazolopyrimidines which may be substituted in the 2- and/or 3-position. JP 2002-308878A and JP 2002-308879A disclose pyrazolopyrimidines substituted in the 2-position. The compounds described in the publications mentioned are known for controlling harmful fungi.

However, the action of the known compounds is in many cases unsatisfactory. It is an object of the present invention to provide compounds having improved activity and/or a broader activity spectrum.

We have found that this object is achieved by the compounds defined at the outset. Furthermore, we have found processes and intermediates for preparing these compounds, compositions comprising them and methods for controlling harmful fungi using the compounds I.

The compounds according to the invention differ from the compounds known from EP-A 71 792, from the compounds described in WO 02/48151 and from the compounds known from JP 2002-308878A and JP 2002-308879A by the substitution of the 7-amino group, by the substitution in the 2- and/or 3-position of the pyrazolopyrimidine skeleton and by the substituents in the 5-position, respectively.

The compounds of the formula I have higher activity against harmful fungi than the known compounds.

The compounds according to the invention can be obtained by different routes. In general, they are obtained from substituted aminopyrazole derivatives II and 2-phenylmalonates III under the conditions known from WO 02/48151. In formula III, R is a C₁-C₄-alkyl group, in particular methyl or ethyl. Some of the pyrazoles II are commercially available or can be prepared under generally known conditions. The preparation of the starting materials III is advantageously carried out under the conditions known from EP-A 10 02 788.

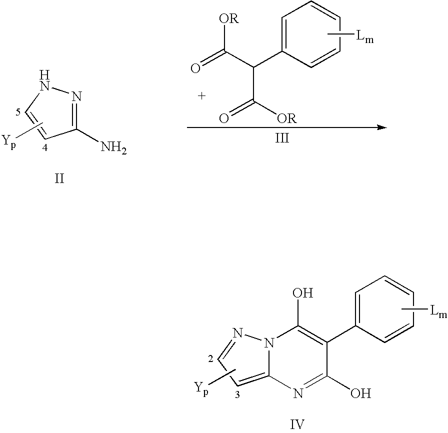

The 5,7-dihydroxy-6-phenylpyrazolopyrimidine IV obtained in this manner are reacted with halogenating agents [HAL] to give 7-halopyrazolopyrimidines of the formula V.

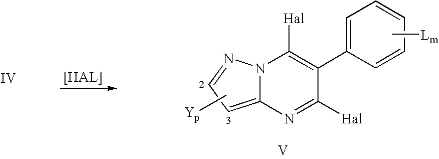

In the formulae IV and V, the definition of the variables corresponds to that for formula I and “Hal” is a halogen atom, preferably bromine or chlorine.

Preference is given to chlorinating or brominating agents such as phosphorus oxybromide, phosphorus oxychloride, thionyl chloride, thionyl bromide or sulfuryl chloride. The reaction can be carried out in the absence or presence of a solvent. Customary reaction temperatures are from 0 to 150° C. or, preferably, from 80 to 125° C.

The compounds of the formula V give, by reaction with amines of the formula VI, pyrazolopyrimidines of the formula I in which X is halogen. They are a preferred subject matter of the invention. Particular preference is given to pyrazolopyrimidines which carry a group Y in the 3-position.

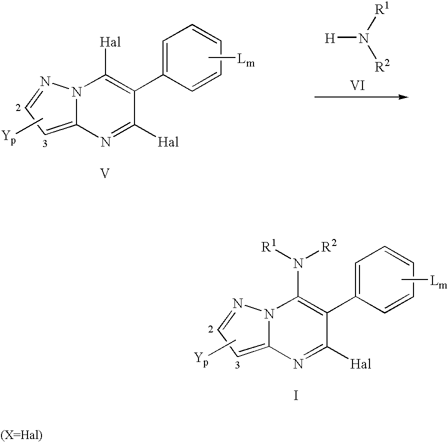

The reaction of V with amines VI is advantageously carried out at from 0° C. to 70° C., preferably from 10° C. to 35° C., with preference in the presence of an inert solvent, such as an ether, for example dioxane, diethyl ether or, in particular, tetrahydrofuran, halogenated hydrocarbons, such as dichloromethane, and aromatic hydrocarbons, such as, for example, toluene [cf. WO 98/46608; WO 02/48151].

Preference is given to using a base, such as tertiary amines, for example triethylamine, or inorganic bases, such as potassium carbonate; it is also possible for excess amine of the formula IV to serve as base.

Compounds of the formula I which carry a group Y in the 3-position are advantageously obtained from 5,7-dihydroxypyrazolopyrimidines VII by reaction with a halogenating agent. The trihalopyrazolopyrimidines of the formula VIII obtainable in this manner are useful intermediates for preparing the compounds of the formula I.

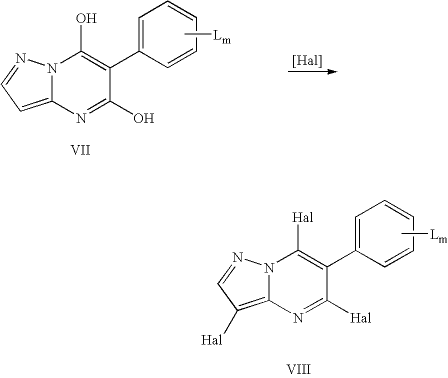

In the formulae VII and VIII, the definition of the variables corresponds to that for formula I and “Hal” is a halogen atom, preferably bromine or chlorine. The halogenation is carried out analogously to that of the compounds IV.

By condensation with amines VI, it is possible to obtain the compounds I.A under the conditions described above from the compounds VIII. They are a particularly preferred subject matter of the invention. Compounds I.A are also useful intermediates for preparing further compounds I.

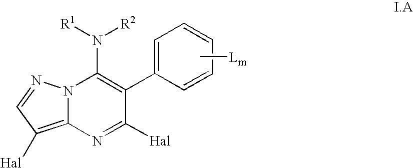

5,7-Dihydroxypyrazolopyrimidines of the formula II are known from WO 02/48151. Some of the amines of the formula VI are known, are commercially available or can be prepared by known methods.

Compounds I in which X in the 5-position is cyano, C₁-C₆-alkoxy or C₁-C₂-haloalkoxy (formula I.B) can be prepared in an advantageous manner from starting materials of the formula I.A, by the routes illustrated below.

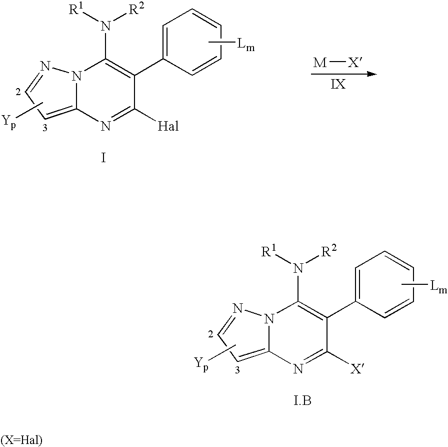

Compounds I (X=Hal) are reacted with compounds M-X′ (formula IX) to give compounds I.B. Depending on the meaning of the group X′ to be introduced, the compounds IX are an inorganic cyanide or an alkoxide. The reaction is advantageously carried out in the presence of an inert solvent. In formula IX, the cation M is of minor importance; for practical reasons, preference is usually given to ammonium, tetraalkylammonium or alkali metal or alkaline earth metal salts. The reaction temperature is usually from 0 to 120° C., preferably from 10 to 40° C. [cf. J. Heterocycl. Chem. 12 (1975), pp. 861-863].

Suitable solvents include ethers, such as dioxane, diethyl ether and, preferably, tetrahydrofuran, halogenated hydrocarbons, such as dichloromethane, and aromatic hydrocarbons, such as toluene.

Compounds I in which X is C₁-C₄-alkyl or C₁-C₄-haloalkyl (formula I.C) can advantageously be prepared from compounds of the formula I, in which X is halogen, by the routes illustrated below.

Compounds of the formula I.C in which X″ is C₁-C₄-alkyl can be obtained by coupling 5-halotriazolopyrimidines of the formula IV.A with organometallic reagents of the formula X. In one embodiment of this process, the reaction is carried out under transition metal catalysis, such as Ni- or Pd catalysis.

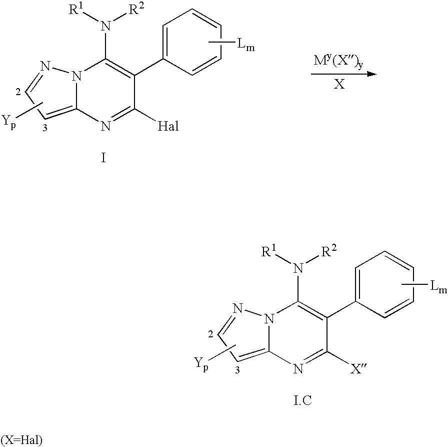

In formula X, X″ is C₁-C₄-alkyl and M is a metal ion of the valency Y, such as, for example, B, Zn or Sn. This reaction can be carried out, for example, analogously to the following methods: J. Chem. Soc. Perkin Trans. I, 1187 (1994), ibid 1, 2345 (1996); WO 99/41255; Aust. J. Chem., 43 (1990), 733; J. Org. Chem., 43 (1978), 358; J. Chem. Soc. Chem. Commun. (1979), 866; Tetrahedron Lett., 34 (1993), 8267; ibid 33 (1992), 413.

Compounds of the formula I in which X is C₁-C₄-alkyl or C₁-C₄-haloalkyl (formula I.C) can advantageously also be obtained by the synthesis route below:

The 5-alkyl-7-hydroxy-6-phenylpyrazolopyrimidines XII are obtained from substituted aminopyrazole derivatives II and the ketoesters XI. In formula XI, R is a C₁-C₄-alkyl group, in particular methyl or ethyl, and X″ is C₁-C₄-alkyl. Using the easily obtainable 2-phenylacetoacetic esters XIa where X″═CH₃, 5-methyl-7-hydroxy-6-phenylpyrazolo-pyrimidines are obtained [cf. Chem. Pharm. Bull., 9 (1961), 801]. The starting compounds XI are advantageously prepared under the conditions known from EP-A 10 02 788.

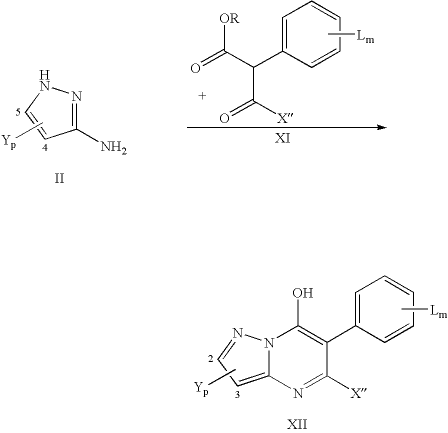

The 5-alkyl-7-hydroxy-6-phenylpyrazolopyrimidines XII obtained in this manner are reacted analogously to compounds IV with halogenating agents [HAL] to give 7-halopyrazolopyrimidines of the formula XIII.

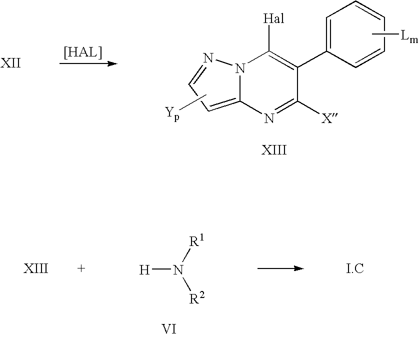

The reaction of XIII with amines VI is carried out analogously to the reaction of the compounds V with VI.

Alternatively, compounds of the formula I.C can also be prepared from compounds I.A and malonates of the formula XIV. In formula XIV, X′″ is hydrogen or C₁-C₃-alkyl and R is C₁-C₄-alkyl. These compounds are converted into compounds of the formula XV and decarboxylated to give compounds I.C [cf. U.S. Pat. No. 5 994 360].

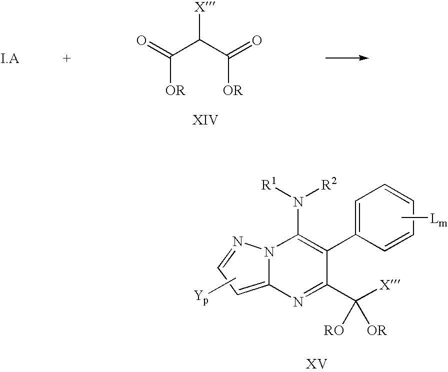

The malonates XIV are known from the literature [J. Am. Chem. Soc. 64 (1942), 2714; J. Org. Chem. 39 (1974), 2172; Helv. Chim. Acta 61 (1978), 1565] or they can be prepared in accordance with the literature cited.

The subsequent hydrolysis of the ester XV is carried out under generally customary conditions; depending on the different structural elements, -alkaline or acidic hydrolysis of the compounds XV may be advantageous. Partial or complete decarboxylation to I.C may even take place under the conditions of the ester hydrolysis.

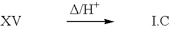

Usually, decarboxylation takes place at temperatures of from 20° C. to 180° C., preferably from 50° C. to 120° C., in an inert solvent, if appropriate in the presence of an acid.

Compounds of the formula I in which x is CN or C₁-C₄-alkoxy (formula I.D) can be obtained by the synthesis route below:

By selective hydrolysis, 5,7-dihalopyrazolopyrimidines of the formula V are converted into 5-halo-7-hydroxypyrazolopyrimidines of the formula Va, analogously to Chem. Pharm. Bull. 1961, 9801 (triazolopyrimidines) or J. Agric. Food Chem. 41, 12, 1993, 2411 (pyrimidines), or by acid catalysis using 10% strength HCl in dioxane in accordance with Khim. Geterotsikl. Soedin, RU, 21, 3, 1985, 378 (pyrimidines).

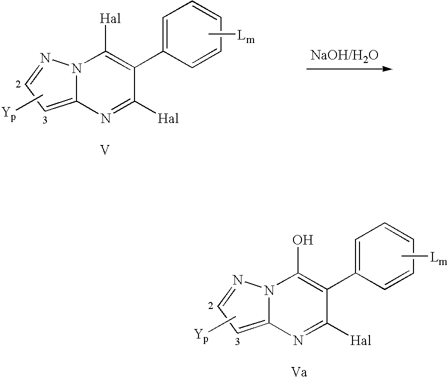

Using organometallic reagents of the formula IX, the 5-halo-7-hydroxypyrazolo-pyrimidines Va obtained in this manner are converted into the compounds XIIa.

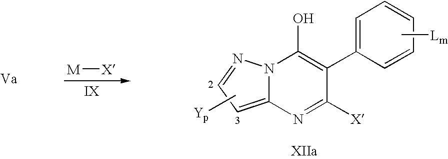

in which X′ is CN or C₁-C₄-alkoxy,

followed by halogenation of XIIa to give compounds of the formula XIIIa

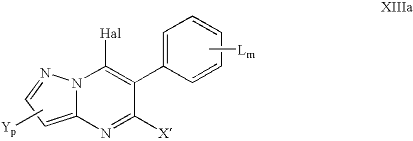

and reaction of XIIIa with amines of the formula VI to give compounds of the formula I.D., analogously to the preparation of the compounds I.A.

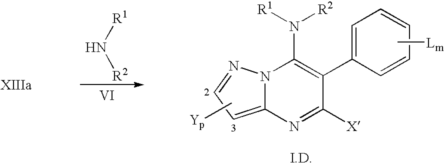

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of the symbols given in the formulae above, collective terms were used which are generally representative of the following substituents:

halogen: fluorine, chlorine, bromine and iodine;

alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4 or 6 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl; alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated straight-chain or branched hydrocarbon radicals having 2 to 10 carbon atoms and one or two double bonds in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- to ten-membered saturated, partially unsaturated or aromatic heterocycle which contains one to four heteroatoms from the group consisting of O, N and S:

5- or 6-membered partially unsaturated heterocyclyl which contains one to three nitrogen atoms and/or one oxygen or sulfur atom or one or two oxygen and/or sulfur atoms, for example 3,6-dihydro-2H-pyridin-1-yl and 2,5-dihydropyrrol-1-yl.

5- or 6-membered saturated heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;

5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;

6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

Alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂; oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;

oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

The particularly preferred embodiments of the intermediates with respect to the variables correspond to those of radicals Lₘ, R¹, R², X and Yₚ of formula I.

With a view to the intended use of the pyrazolopyrimidines of the formula I, particular preference is given to the following meanings of the substituents, in each case on their own or in combination:

Preference is given to compounds I in which R¹ is C₁-C₄-alkyl, C₂-C₆-alkenyl or C₁-C₈-haloalkyl.

Particular preference is given to compounds I in which R¹ is an alkenyl or alkynyl group which is branched at the a carbon atom. In these cases, group R¹ corresponds to a group A:

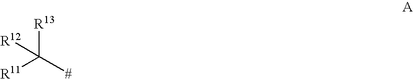

in which # is the bond to the nitrogen atom and

R¹¹ is C₁-C₃-alkyl or C₁-C₃-haloalkyl;

R¹² is hydrogen, C₁-C₃-alkyl or C₁-C₃-haloalkyl;

R¹³ is C₂-C₁₀-alkenyl or C₂-C₈-alkynyl, where R¹³ may be unsubstituted or partially or fully halogenated and/or may carry one to three groups R^{a}.

Preference is likewise given to compounds I in which R¹ is a 5- or 6-membered saturated or aromatic heterocycle which contains one or two heteroatoms from the group consisting of N, O and S and which may be substituted by one or two alkyl or haloalkyl groups.

Preference is given to compounds I in which R¹ is a group B:

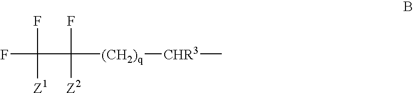

in which

Z¹ is hydrogen, fluorine or C₁-C₆-fluoroalkyl,

Z² is hydrogen or fluorine, or

Z¹ and Z² together form a double bond;

q is 0 or 1; and

R³ is hydrogen or methyl.

Moreover, preference is given to compounds I in which R¹ is C₃-C₆-cycloalkyl which may be substituted by C₁-C₄-alkyl.

Particular preference is given to compounds I in which R² is hydrogen.

Likewise, preference is given to compounds I in which R² is methyl or ethyl.

If R¹ and/or R² comprise haloalkyl or haloalkenyl groups having a center of chirality, the (S)-isomers are preferred for these groups. In the case of halogen-free alkyl or alkenyl groups having a center of chirality in R¹ or R², preference is given to the (R)-configured isomers.

Preference is furthermore given to compounds I in which R¹ and R² together with the nitrogen atom to which they are attached form a saturated or unsaturated five- or six-membered ring which may be interrupted by an atom from the group consisting of O, N and S and/or which may carry one or more substituents from the group consisting of halogen, C₁-C₆-alkyl, C₁-C₆-haloalkyl and oxy-C₁-C₃-alkyleneoxy or in which two adjacent ring members may be linked by a C₁-C₄-alkylene chain.

Particular preference is given to compounds I in which R¹ and R² together with the nitrogen atom to which they are attached form a piperidinyl, morpholinyl or thiomorpholinyl ring, in particular a piperidinyl ring which is unsubstituted or substituted by one to three halogen, C₁-C₄-alkyl or C₁-C₄-haloalkyl groups, in particular by 4-methyl.

A further preferred subject of the invention is compounds I in which R¹ and R² together with the nitrogen atom to which they are attached form a pyrazole ring which is unsubstituted or substituted by one or two halogen, C₁-C₄-alkyl or C₁-C₄-haloalkyl groups, in particular by 3,5-dimethyl or 3,5-di(trifluoromethyl).

Preference is given to compounds I in which at least one group L is located ortho to the point of attachment to the triazolopyrimidine skeleton; in particular to those compounds, in which n has the value 1, 2 or 3.

Preference is given to compounds I in which Lₘ is halogen, methyl, ethyl, C₁-haloalkyl, methoxy or —C(═O)-A², where A² is hydrogen, hydroxyl, C₁-C₄-alkoxy, C₁-C₄-haloalkoxy, C₁-C₂-alkylamino or di-C₁-C₂-alkylamino.

Moreover, particular preference is given to compounds I in which the phenyl group substituted by Lₘ is the group C

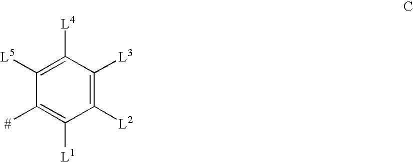

in which # is the point of attachment to the pyrazolopyrimidine skeleton and

L¹ is fluorine, chlorine, CH₃ or CF₃;

L²,L⁴ independently of one another are hydrogen or fluorine;

L³ is hydrogen, fluorine, chlorine, cyano, CH₃ or COOCH₃; and

L⁵ is hydrogen, fluorine or CH₃.

Preference is given to compounds I in which X is halogen, CN, OH, C₁-C₄-alkyl or C₁-C₄-alkoxy.

Further preference is given to compounds I in which X is halogen, CN or C₁-C₄-alkyl.

Particular preference is given to compounds I in which X is halogen or C₁-C₄-alkyl, such as chlorine or methyl, in particular halogen, such as chlorine.

In addition, preference is given to compounds I in which Y is halogen, in particular fluorine or chlorine, or alkyl, in particular methyl. In a particularly preferred embodiment of the invention, the group X is a chlorine atom and Y is fluorine, chlorine or methyl.

Preference is furthermore given to compounds I in which the index p=1.

In addition, preference is given to compounds I in which the group Y is located in the 3-position of the pyrimidine skeleton (formula I.1):

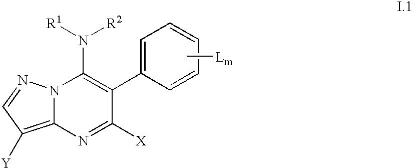

In another preferred embodiment of the invention, group Y is located in the 2-position of the pyrimidine skeleton (formula I.2):

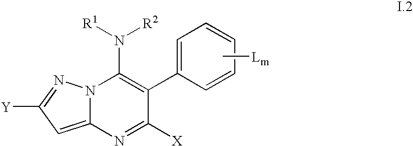

Compounds of the formula I.A are a particularly preferred subject matter of the invention:

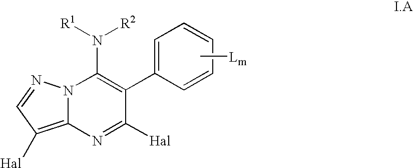

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

Table 1

Compounds of the formula I.1 in which X and Y are chlorine, Lₘ is 2-fluoro-6-chloro and the combination of R¹ and R² corresponds for each compound to one row of Table A

Table 2

Compounds of the formula I.1 in which X and Y are chlorine, Lₘ is 2,6-difluoro and the combination of R¹ and R² corresponds for each compound to one row of Table A

Table 3

Compounds of the formula I.1 in which X and Y are chlorine, Lₘ is 2,6-dichloro and the combination of R¹ and R² corresponds for each compound to one row of Table A

Table 4

Compounds of the formula I.1 in which X and Y are chlorine, Lₘ is 2-fluoro-6-methyl and the combination of R¹ and R² corresponds for each compound to one row of Table A

Table 5

Compounds of the formula I.1 in which X and Y are chlorine, Lₘ is 2,4,6-trifluoro and the combination of R¹ and R² corresponds for each compound to one row of Table A

Table 6

Compounds of the formula I.1 in which X and Y are chlorine, Lₘ is 2,6-difluoro-4-methoxy and the combination of R¹ and R² corresponds for each compound to one row of Table A

Table 7

Compounds of the formula I.1 in which X and Y are chlorine, Lₘ is pentafluoro and the combination of R¹ and R² corresponds for each compound to one row of Table A

Table 8

Compounds of the formula I.1 in which X and Y are chlorine, Lₘ is 2-methyl-4-fluoro and the combination of R¹ and R² corresponds for each compound to one row of Table A

Table 9

Compounds of the formula I.1 in which X and Y are chlorine, Lₘ is 2-trifluoromethyl and the combination of R¹ and R² corresponds for each compound to one row of Table A

Table 10

Compounds of the formula I.1 in which X and Y are chlorine, Lₘ is 2-methoxy-6-fluoro and the combination of R¹ and R² corresponds for each compound to one row of Table A

Table 11

Compounds of the formula I.1 in which X and Y are chlorine, Lₘ is 2-chloro and the combination of R¹ and R² corresponds for each compound to one row of Table A

Table 12

Compounds of the formula I.1 in which X and Y are chlorine, Lₘ is 2-fluoro and the combination of R¹ and R² corresponds for each compound to one row of Table A

Table 13

Compounds of the formula I.1 in which X and Y are chlorine, Lₘ is 2,4-difluoro and the combination of R¹ and R² corresponds for each compound to one row of Table A

Table 14

Compounds of the formula I.1 in which X and Y are chlorine, Lₘ is 2-fluoro4-chloro and the combination of R¹ and R² corresponds for each compound to one row of Table A

Table 15

Compounds of the formula I.1 in which X and Y are chlorine, Lₘ is 2-chloro-4-fluoro and the combination of R¹ and R² corresponds for each compound to one row of Table A

Table 16

Compounds of the formula I.1 in which X and Y are chlorine, Lₘ is 2,3-difluoro and the combination of R¹ and R² corresponds for each compound to one row of Table A

Table 17

Compounds of the formula I.1 in which X and Y are chlorine, Lₘ is 2,5-difluoro and the combination of R¹ and R² corresponds for each compound to one row of Table A

Table 18

Compounds of the formula I.1 in which X and Y are chlorine, Lₘ is 2,3,4-trifluoro and the combination of R¹ and R² corresponds for each compound to one row of Table A

Table 19

Compounds of the formula I.1 in which X and Y are chlorine, Lₘ is 2-methyl and the combination of R¹ and R² corresponds for each compound to one row of Table A

Table 20

Compounds of the formula I.1 in which X and Y are chlorine, Lₘ is 2,4-dimethyl and the combination of R¹ and R² corresponds for each compound to one row of Table A

Table 21

Compounds of the formula I.1 in which X and Y are chlorine, Lₘ is 2-methyl-4-chloro and the combination of R¹ and R² corresponds for each compound to one row of Table A

Table 22

Compounds of the formula I.1 in which X and Y are chlorine, Lₘ is 2-fluoro-4-methyl and the combination of R¹ and R² corresponds for each compound to one row of Table A

Table 23

Compounds of the formula I.1 in which X and Y are chlorine, Lₘ is 2,6-dimethyl and the combination of R¹ and R² corresponds for each compound to one row of Table A

Table 24

Compounds of the formula I.1 in which X and Y are chlorine, Lₘ is 2,4,6-trimethyl and the combination of R¹ and R² corresponds for each compound to one row of Table A

Table 25

Compounds of the formula I.1 in which X and Y are chlorine, Lₘ is 2,6-difluoro-4-cyano and the combination of R¹ and R² corresponds for each compound to one row of Table A

Table 26

Compounds of the formula I.1 in which X and Y are chlorine, Lₘ is 2,6-difluoro-4-methyl and the combination of R¹ and R² corresponds for each compound to one row of Table A

Table 27

Compounds of the formula I.1 in which X and Y are chlorine, Lₘ is 2,6-difluoro-4-methoxycarbonyl and the combination of R¹ and R² corresponds for each compound to one row of Table A

Table 28

Compounds of the formula I.1 in which X and Y are chlorine, Lₘ is 2-trifluoromethyl-4-fluoro and the combination of R¹ and R² corresponds for each compound to one row of Table A

Table 29

Compounds of the formula I.1 in which X and Y are chlorine, Lₘ is 2-trifluoromethyl-5-fluoro and the combination of R¹ and R² corresponds for each compound to one row of Table A

Table 30

Compounds of the formula I.1 in which X and Y are chlorine, Lₘ is 2-trifluoromethyl-5-chloro and the combination of R¹ and R² corresponds for each compound to one row of Table A

Table 31

Compounds of the formula I.2 in which X is chlorine, Y is methyl, Lₘ is 2-fluoro-6-chloro and the combination of R¹ and R² corresponds for each compound to one row of Table A

Table 32

Compounds of the formula I.2 in which X is chlorine, Y is methyl, Lₘ is 2,6-difluoro and the combination of R¹ and R² corresponds for each compound to one row of Table A

Table 33

Compounds of the formula I.2 in which X is chlorine, Y is methyl, Lₘ is 2,6-dichloro and the combination of R¹ and R² corresponds for each compound to one row of Table A

Table 34

Compounds of the formula I.2 in which X is chlorine, Y is methyl, Lₘ is 2-fluoro-6-methyl and the combination of R¹ and R² corresponds for each compound to one row of Table A

Table 35

Compounds of the formula I.2 in which X is chlorine, Y is methyl, Lₘ is 2,4,6-trifluoro and the combination of R¹ and R² corresponds for each compound to one row of Table A

Table 36

Compounds of the formula I.2 in which X is chlorine, Y is methyl, Lₘ is 2,6-difluoro-4-methoxy and the combination of R¹ and R² corresponds for each compound to one row of Table A

Table 37

Compounds of the formula I.2 in which X is chlorine, Y is methyl, Lₘ is pentafluoro and the combination of R¹ and R² corresponds for each compound to one row of Table A

Table 38

Compounds of the formula I.2 in which X is chlorine, Y is methyl, Lₘ is 2-methyl-4-fluoro and the combination of R¹ and R² corresponds for each compound to one row of Table A

Table 39

Compounds of the formula I.2 in which X is chlorine, Y is methyl, Lₘ is 2-trifluoromethyl and the combination of R¹ and R² corresponds for each compound to one row of Table A

Table 40

Compounds of the formula I.2 in which X is chlorine, Y is methyl, Lₘ is 2-methoxy-6-fluoro and the combination of R¹ and R² corresponds for each compound to one row of Table A

Table 41

Compounds of the formula I.2 in which X is chlorine, Y is methyl, Lₘ is 2-chloro and the combination of R¹ and R² corresponds for each compound to one row of Table A

Table 42

Compounds of the formula I.2 in which X is chlorine, Y is methyl, Lₘ is 2-fluoro and the combination of R¹ and R² corresponds for each compound to one row of Table A

Table 43

Compounds of the formula I.2 in which X is chlorine, Y is methyl, Lₘ is 2,4-difluoro and the combination of R¹ and R² corresponds for each compound to one row of Table A

Table 44

Compounds of the formula I.2 in which X is chlorine, Y is methyl, Lₘ is 2-fluoro-4-chloro and the combination of R¹ and R² corresponds for each compound to one row of Table A

Table 45

Compounds of the formula I.2 in which X is chlorine, Y is methyl, Lₘ is 2-chloro-4-fluoro and the combination of R¹ and R² corresponds for each compound to one row of Table A

Table 46

Compounds of the formula I.2 in which X is chlorine, Y is methyl, Lₘ is 2,3-difluoro and the combination of R¹ and R² corresponds for each compound to one row of Table A

Table 47

Compounds of the formula I.2 in which X is chlorine, Y is methyl, Lₘ is 2,5-difluoro and the combination of R¹ and R² corresponds for each compound to one row of Table A

Table 48

Compounds of the formula I.2 in which X is chlorine, Y is methyl, Lₘ is 2,3,4-trifluoro and the combination of R¹ and R² corresponds for each compound to one row of Table A

Table 49

Compounds of the formula I.2 in which X is chlorine, Y is methyl, Lₘ is 2-methyl and the combination of R¹ and R² corresponds for each compound to one row of Table A

Table 50

Compounds of the formula I.2 in which X is chlorine, Y is methyl, Lₘ is 2,4-dimethyl and the combination of R¹ and R² corresponds for each compound to one row of Table A

Table 51

Compounds of the formula I.2 in which X is chlorine, Y is methyl, Lₘ is 2-methyl-4-chloro and the combination of R¹ and R² corresponds for each compound to one row of Table A

Table 52

Compounds of the formula I.2 in which X is chlorine, Y is methyl, Lₘ is 2-fluoro-4-methyl and the combination of R¹ and R² corresponds for each compound to one row of Table A

Table 53

Compounds of the formula I.2 in which X is chlorine, Y is methyl, Lₘ is 2,6-dimethyl and the combination of R¹ and R² corresponds for each compound to one row of Table A.

Table 54

Compounds of the formula I.2 in which X is chlorine, Y is methyl, Lₘ is 2,4,6-trimethyl and the combination of R¹ and R² corresponds for each compound to one row of Table A

Table 55

Compounds of the formula I.2 in which X is chlorine, Y is methyl, Lₘ is 2,6-difluoro-4-cyano and the combination of R¹ and R² corresponds for each compound to one row of Table A

Table 56

Compounds of the formula I.2 in which X is chlorine, Y is methyl, Lₘ is 2,6-difluoro-4-methyl and the combination of R¹ and R² corresponds for each compound to one row of Table A

Table 57

Compounds of the formula I.2 in which X is chlorine, Y is methyl, Lₘ is 2,6-difluoro-4-methoxycarbonyl and the combination of R¹ and R² corresponds for each compound to one row of Table A

Table 58

Compounds of the formula I.2 in which X is chlorine, Y is methyl, Lₘ is 2-trifluoromethyl-4-fluoro and the combination of R¹ and R² corresponds for each compound to one row of Table A

Table 59

Compounds of the formula I.2 in which X is chlorine, Y is methyl, Lₘ is 2-trifluoromethyl-5-fluoro and the combination of R¹ and R² corresponds for each compound to one row of Table A

Table 60

Compounds of the formula I.2 in which X is chlorine, Y is methyl, Lₘ is 2-trifluoromethyl-5-chloro and the combination of R¹ and R² corresponds for each compound to one row of Table A

Table 61

Compounds of the formula I.3, in which X is methyl, Y is hydrogen, Lₘ is 2-fluoro-6-chloro and the combination of R¹ and R² corresponds for each compound to one row of Table A

Table 62

Compounds of the formula I.3, in which X is methyl, Y is hydrogen, Lₘ is 2,6-difluoro and the combination of R¹ and R² corresponds for each compound to one row of Table A

Table 63

Compounds of the formula I.3, in which X is methyl, Y is hydrogen, Lₘ is 2,6-dichloro and the combination of R¹ and R² corresponds for each compound to one row of Table A

Table 64

Compounds of the formula I.3, in which X is methyl, Y is hydrogen, Lₘ is 2-fluoro-6-methyl and the combination of R¹ and R² corresponds for each compound to one row of Table A

Table 65

Compounds of the formula I.3, in which X is methyl, Y is hydrogen, Lₘ is 2,4,6-trifluoro and the combination of R¹ and R² corresponds for each compound to one row of Table A

Table 66

Compounds of the formula I.3, in which X is methyl, Y is hydrogen, Lₘ is 2,6-difluoro-4-methoxy and the combination of R¹ and R² corresponds for each compound to one row of Table A

Table 67

Compounds of the formula I.3, in which X is methyl, Y is hydrogen, Lₘ is pentafluoro and the combination of R¹ and R² corresponds for each compound to one row of Table A

Table 68

Compounds of the formula I.3, in which X is methyl, Y is hydrogen, Lₘ is 2-methyl-4-fluoro and the combination of R¹ and R² corresponds for each compound to one row of Table A

Table 69

Compounds of the formula I.3, in which X is methyl, Y is hydrogen, Lₘ is 2-trifluoromethyl and the combination of R¹ and R² corresponds for each compound to one row of Table A

Table 70

Compounds of the formula I.3, in which X is methyl, Y is hydrogen, Lₘ is 2-methoxy-6-fluoro and the combination of R¹ and R² corresponds for each compound to one row of Table A

Table 71

Compounds of the formula I.3, in which X is methyl, Y is hydrogen, Lₘ is 2-chloro and the combination of R¹ and R² corresponds for each compound to one row of Table A

Table 72

Compounds of the formula I.3, in which X is methyl, Y is hydrogen, Lₘ is 2-fluoro and the combination of R¹ and R² corresponds for each compound to one row of Table A

Table 73

Compounds of the formula I.3, in which X is methyl, Y is hydrogen, Lₘ is 2,4-difluoro and the combination of R¹ and R² corresponds for each compound to one row of Table A

Table 74

Compounds of the formula I.3, in which X is methyl, Y is hydrogen, Lₘ is 2-fluoro-4-chloro and the combination of R¹ and R² corresponds for each compound to one row of Table A

Table 75

Compounds of the formula I.3, in which X is methyl, Y is hydrogen, Lₘ is 2-chloro-4-fluoro and the combination of R¹ and R² corresponds for each compound to one row of Table A

Table 76

Compounds of the formula I.3, in which X is methyl, Y is hydrogen, Lₘ is 2,3-difluoro and the combination of R¹ and R² corresponds for each compound to one row of Table A

Table 77

Compounds of the formula I.3, in which X is methyl, Y is hydrogen, Lₘ is 2,5-difluoro and the combination of R¹ and R² corresponds for each compound to one row of Table A

Table 78

Compounds of the formula I.3, in which X is methyl, Y is hydrogen, Lₘ is 2,3,4-trifluoro and the combination of R¹ and R² corresponds for each compound to one row of Table A

Table 79

Compounds of the formula I.3, in which X is methyl, Y is hydrogen, Lₘ is 2-methyl and the combination of R¹ and R² corresponds for each compound to one row of Table A

Table 80

Compounds of the formula I.3, in which X is methyl, Y is hydrogen, Lₘ is 2,4-dimethyl and the combination of R¹ and R² corresponds for each compound to one row of Table A

Table 81

Compounds of the formula I.3, in which X is methyl, Y is hydrogen, Lₘ is 2-methyl-4-chloro and the combination of R¹ and R² corresponds for each compound to one row of Table A

Table 82

Compounds of the formula I.3, in which X is methyl, Y is hydrogen, Lₘ is 2-fluoro-4-methyl and the combination of R¹ and R² corresponds for each compound to one row of Table A

Table 83

Compounds of the formula I.3, in which X is methyl, Y is hydrogen, Lₘ is 2,6-dimethyl and the combination of R¹ and R² corresponds for each compound to one row of Table A

Table 84

Compounds of the formula I.3, in which X is methyl, Y is hydrogen, Lₘ is 2,4,6-trimethyl and the combination of R¹ and R² corresponds for each compound to one row of Table A

Table 85

Compounds of the formula I.3, in which X is methyl, Y is hydrogen, Lₘ is 2,6-difluoro-4-cyano and the combination of R¹ and R² corresponds for each compound to one row of Table A

Table 86

Compounds of the formula I.3, in which X is methyl, Y is hydrogen, Lₘ 2,6-difluoro-4-methyl and the combination of R¹ and R² corresponds for each compound to one row of Table A

Table 87

Compounds of the formula I.3, in which X is methyl, Y is hydrogen, Lₘ is 2,6-difluoro-4-methoxycarbonyl and the combination of R¹ and R² corresponds for each compound to one row of Table A

Table 88

Compounds of the formula I.3, in which X is methyl, Y is hydrogen, Lₘ is 2-trifluoromethyl-4-fluor and the combination of R¹ and R² corresponds for each compound to one row of Table A

Table 89

Compounds of the formula I.3, in which X is methyl, Y is hydrogen, Lₘ is 2-trifluoromethyl-5-fluor and the combination of R¹ and R² corresponds for each compound to one row of Table A

Table 90

Compounds of the formula I.3, in which X is methyl, Y is hydrogen, Lₘ is 2-trifluoromethyl-5-chlor and the combination of R¹ and R² corresponds for each compound to one row of Table A

TABLE A

| No. | R¹ | R² |
|---|---|---|
| A-1 | CH₃ | H |
| A-2 | CH₃ | CH₃ |
| A-3 | CH₂CH₃ | H |
| A-4 | CH₂CH₃ | CH₃ |
| A-5 | CH₂CH₃ | CH₂CH₃ |
| A-6 | CH₂CF₃ | H |
| A-7 | CH₂CF₃ | CH₃ |
| A-8 | CH₂CF₃ | CH₂CH₃ |
| A-9 | CH₂CCl₃ | H |
| A-10 | CH₂CCl₃ | CH₃ |
| A-11 | CH₂CCl₃ | CH₂CH₃ |
| A-12 | CH₂CH₂CH₃ | H |
| A-13 | CH₂CH₂CH₃ | CH₃ |
| A-14 | CH₂CH₂CH₃ | CH₂CH₃ |
| A-15 | CH₂CH₂CH₃ | CH₂CH₂CH₃ |
| A-16 | CH(CH₃)₂ | H |
| A-17 | CH(CH₃)₂ | CH₃ |
| A-18 | CH(CH₃)₂ | CH₂CH₃ |
| A-19 | CH₂CH₂CH₂CH₃ | H |
| A-20 | CH₂CH₂CH₂CH₃ | CH₃ |
| A-21 | CH₂CH₂CH₂CH₃ | CH₂CH₃ |
| A-22 | CH₂CH₂CH₂CH₃ | CH₂CH₂CH₃ |
| A-23 | CH₂CH₂CH₂CH₃ | CH₂CH₂CH₂CH₃ |
| A-24 | (±) CH(CH₃)—CH₂CH₃ | H |
| A-25 | (±) CH(CH₃)—CH₂CH₃ | CH₃ |
| A-26 | (±) CH(CH₃)—CH₂CH₃ | CH₂CH₃ |
| A-27 | (S) CH(CH₃)—CH₂CH₃ | H |
| A-28 | (S) CH(CH₃)—CH₂CH₃ | CH₃ |
| A-29 | (S) CH(CH₃)—CH₂CH₃ | CH₂CH₃ |
| A-30 | (R) CH(CH₃)—CH₂CH₃ | H |
| A-31 | (R) CH(CH₃)—CH₂CH₃ | CH₃ |
| A-32 | (R) CH(CH₃)—CH₂CH₃ | CH₂CH₃ |
| A-33 | (±) CH(CH₃)—CH(CH₃)₂ | H |
| A-34 | (±) CH(CH₃)—CH(CH₃)₂ | CH₃ |
| A-35 | (±) CH(CH₃)—CH(CH₃)₂ | CH₂CH₃ |
| A-36 | (S) CH(CH₃)—CH(CH₃)₂ | H |
| A-37 | (S) CH(CH₃)—CH(CH₃)₂ | CH₃ |
| A-38 | (S) CH(CH₃)—CH(CH₃)₂ | CH₂CH₃ |
| A-39 | (R) CH(CH₃)—CH(CH₃)₂ | H |
| A-40 | (R) CH(CH₃)—CH(CH₃)₂ | CH₃ |
| A-41 | (R) CH(CH₃)—CH(CH₃)₂ | CH₂CH₃ |
| A-42 | (±) CH(CH₃)—C(CH₃)₃ | H |
| A-43 | (±) CH(CH₃)—C(CH₃)₃ | CH₃ |
| A-44 | (±) CH(CH₃)—C(CH₃)₃ | CH₂CH₃ |
| A-45 | (S) CH(CH₃)—C(CH₃)₃ | H |
| A-46 | (S) CH(CH₃)—C(CH₃)₃ | CH₃ |
| A-47 | (S) CH(CH₃)—C(CH₃)₃ | CH₂CH₃ |
| A-48 | (R) CH(CH₃)—C(CH₃)₃ | H |
| A-49 | (R) CH(CH₃)—C(CH₃)₃ | CH₃ |
| A-50 | (R) CH(CH₃)—C(CH₃)₃ | CH₂CH₃ |
| A-51 | (±) CH(CH₃)—CF₃ | H |
| A-52 | (±) CH(CH₃)—CF₃ | CH₃ |
| A-53 | (±) CH(CH₃)—CF₃ | CH₂CH₃ |
| A-54 | (S) CH(CH₃)—CF₃ | H |
| A-55 | (S) CH(CH₃)—CF₃ | CH₃ |
| A-56 | (S) CH(CH₃)—CF₃ | CH₂CH₃ |
| A-57 | (R) CH(CH₃)—CF₃ | H |
| A-58 | (R) CH(CH₃)—CF₃ | CH₃ |
| A-59 | (R) CH(CH₃)—CF₃ | CH₂CH₃ |
| A-60 | (±) CH(CH₃)—CCl₃ | H |
| A-61 | (±) CH(CH₃)—CCl₃ | CH₃ |
| A-62 | (±) CH(CH₃)—CCl₃ | CH₂CH₃ |
| A-63 | (S) CH(CH₃)—CCl₃ | H |
| A-64 | (S) CH(CH₃)—CCl₃ | CH₃ |
| A-65 | (S) CH(CH₃)—CCl₃ | CH₂CH₃ |
| A-66 | (R) CH(CH₃)—CCl₃ | H |
| A-67 | (R) CH(CH₃)—CCl₃ | CH₃ |
| A-68 | (R) CH(CH₃)—CCl₃ | CH₂CH₃ |
| A-69 | CH₂CF₂CF₃ | H |
| A-70 | CH₂CF₂CF₃ | CH₃ |
| A-71 | CH₂CF₂CF₃ | CH₂CH₃ |
| A-72 | CH₂(CF₂)₂CF₃ | H |
| A-73 | CH₂(CF₂)₂CF₃ | CH₃ |
| A-74 | CH₂(CF₂)₂CF₃ | CH₂CH₃ |
| A-75 | CH₂C(CH₃)═CH₂ | H |
| A-76 | CH₂C(CH₃)═CH₂ | CH₃ |
| A-77 | CH₂C(CH₃)═CH₂ | CH₂CH₃ |
| A-78 | CH₂CH═CH₂ | H |
| A-79 | CH₂CH═CH₂ | CH₃ |
| A-80 | CH₂CH═CH₂ | CH₂CH₃ |
| A-81 | CH(CH₃)CH═CH₂ | H |
| A-82 | CH(CH₃)CH═CH₂ | CH₃ |
| A-83 | CH(CH₃)CH═CH₂ | CH₂CH₃ |
| A-84 | CH(CH₃)C(CH₃)═CH₂ | H |
| A-85 | CH(CH₃)C(CH₃)═CH₂ | CH₃ |
| A-86 | CH(CH₃)C(CH₃)═CH₂ | CH₂CH₃ |
| A-87 | CH₂—C≡CH | H |
| A-88 | CH₂—C≡CH | CH₃ |
| A-89 | CH₂—C≡CH | CH₂CH₃ |
| A-90 | Cyclopentyl | H |
| A-91 | Cyclopentyl | CH₃ |
| A-92 | Cyclopentyl | CH₂CH₃ |
| A-93 | Cyclohexyl | H |
| A-94 | Cyclohexyl | CH₃ |
| A-95 | Cyclohexyl | CH₂CH₃ |
| A-96 | CH₂—C₆H₅ | H |
| A-97 | CH₂—C₆H₅ | CH₃ |
| A-98 | CH₂—C₆H₅ | CH₂CH₃ |

| | |
|---|---|
| A-99 | —(CH₂)₂CH═CHCH₂— |
| A-100 | —(CH₂)₂C(CH₃)═CHCH₂— |
| A-101 | —(CH₂)₂CH(CH₃)(CH₂)₂— |
| A-102 | —(CH₂)₃CHFCH₂— |
| A-103 | —(CH₂)₂CHF(CH₂)₂— |
| A-104 | —CH₂CHF(CH₂)₃— |
| A-105 | —(CH₂)₂CH(CF₃)(CH₂)₂— |
| A-106 | —(CH₂)₂O(CH₂)₂— |
| A-107 | —(CH₂)₂S(CH₂)₂— |
| A-108 | —(CH₂)₅— |
| A-109 | —(CH₂)₄— |
| A-110 | —CH₂CH═CHCH₂— |
| A-111 | —CH(CH₃)(CH₂)₃— |
| A-112 | —CH₂CH(CH₃)(CH₂)₂— |
| A-113 | —CH(CH₃)—(CH₂)₂—CH(CH₃)— |
| A-114 | —CH(CH₃)—(CH₂)₄— |
| A-115 | —CH₂—CH(CH₃)—(CH₂)₃— |
| A-116 | —(CH₂)—CH(CH₃)—CH₂—CH(CH₃)—CH₂— |
| A-117 | —CH(CH₂CH₃)—(CH₂)₄— |
| A-118 | —(CH₂)₂—CHOH—(CH₂)₂— |
| A-119 | —(CH₂)—CH═CH—(CH₂)₂— |
| A-120 | —(CH₂)₆— |
| A-121 | —CH(CH₃)—(CH₂)₅— |
| A-122 | —(CH₂)₂—N(CH₃)—(CH₂)₂— |
| A-123 | —N═CH—CH═CH— |
| A-124 | —N═C(CH₃)—CH═C(CH₃)— |
| A-125 | —N═C(CF₃)—CH═C(CF₃)— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipdaris and Drechslera species on cereals, rice and lawns
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Helminthosporium species on cereals,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally required.

When used in the protection of materials or stored products, the amount of active compound applied depends on the kind of application area and on the desired effect.

Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutyinaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

Products to be Applied Undiluted

H Dustable Powders (DP)

5 parts by weight of a compound according to the invention are ground finely and mixed intimately with 95% of finely divided kaolin. This gives a dustable product.

I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds by appropriate modification of the starting compounds. The compounds thus obtained are listed in the tables below, together with physical data.

### Example 1

### 2-Methyl-6-(2,4,6-trifluorophenyl)pyrazolo[1,5-a]pyrimidine-5,7-diol [1-1]

A mixture of 3 g (10.3 mmol) of diethyl 2-(2,4,6-trifluorophenyl)malonate, 1 g (10.3 mmol) of 3-amino-5-methylpyrazole and 2.11 g (11.4 mmol) of tributylamine was stirred at 140° C. for 5 hours, the ethanol formed being removed by distillation. After cooling to 20-25° C. about 10% strength NaOH solution was added. The mixture was extracted with methyl tert-butyl ether (MTBE) and the aqueous phase was acidified with dil. HCl solution. The resulting precipitated crystals were filtered off and dried. This gave 3 g of the title compound of m.p. 304° C.

### Example 2

### 3,5,7-Trichloro-6-(2,4,6-trifluorophenyl)pyrazolo[1,5-a]pyrimidine [11-1]

3.0 g (10.7 mmol) of 6-(2,4,6-trifluorophenyl)pyrazolo[1,5-a]pyrimidine-5,7-diol (cf. WO 02/48151), 2.22 g (10.7 mmol) of phosphorus pentachloride and 9.72 g (42.6 mmol) of benzyltriethylammonium chloride were refluxed in a solution of 40 ml of phosphoryl chloride and 35 ml of acetonitrile for 20 hours. After cooling, volatile components were removed from the reaction mixture and the residue was taken up in dichloromethane. The solution was poured into ice-water and neutralized with sodium carbonate solution. After phase separation, the organic phase was washed with water and dried, and the solvent was removed. The residue gave, after chromatography on silica gel (cyclohexane/ethyl acetate mixtures), 0.5 g of the title compound of m.p.: 128° C.

### Example 3

### 3,5-Dichloro-7-(4-methylpiperidin-1-yl)-6-(2,4,6-trifluorophenyl)-pyrazolo[1,5-a]pyrimidine[III-1]

A solution of 0.1 g (0.284 mmol) of 3,5,7-trichloro-6-(2,4,6-trifluorophenyl)pyrazolo[1,5-a]pyrimidine (Ex. 2), 0.03 g (0.284 mmol) of 4-methylpiperidine and 0.03 g of triethylamine in 1 ml of dichloromethane was stirred at 20-25° C. for about 4 hours. Water was added and the phases were separated, and the organic phase was then washed with dil. HCl solution and water and dried, and the solvent was removed. The residue gave 0.102 g of the title compound of m.p. 138° C.

### Example 4

### 5-Methyl-7-(4-methylpiperidin-1-yl)-6-(2,4,6-trifluorophenyl)pyrazolo[1,5-a]pyrimidine

### Step a: 5-Methyl-6-(2,4,6-trifluorophenyl)pyrazolo[1,5-a]pyrimidin-7-ol

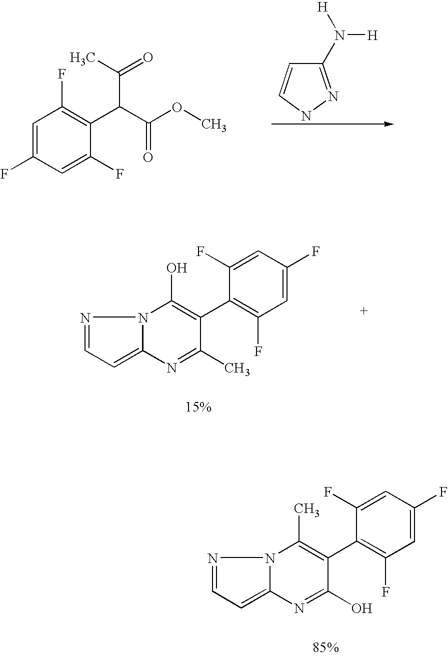

A mixture of 2 g (0.0081 mol) of methyl 3-oxo-2-(2,4,6-trifluorophenyl)butanoate, 0.68 g (0.0081 mol) of 3-aminopyrazole and 1.66 g (0.0089 mol) of tributylamine was stirred at 160° C. for 4 hours, and the methanol formed was distilled off. After cooling, 10% strength sodium hydroxide solution was added. The mixture was extracted with methyl tert-butyl ether, and the aqueous phase was acidified with dilute hydrochloric acid. The crystals which precipitated from the aqueous phase were filtered off and dried. This gave 1.9 g of an isomer mixture, 15% of which consisted of the title compound and 85% of which consisted of 7-methyl-6-(2,4,6-trifluorophenyl)pyrazolo[1,5-a]pyrimidin-5-ol. The isomer mixture was reacted further without purification.

### Step b: 7-Chloro-5-methyl-6-(2,4,6-trifluorophenyl)pyrazolo[1,5-a]pyrimidine

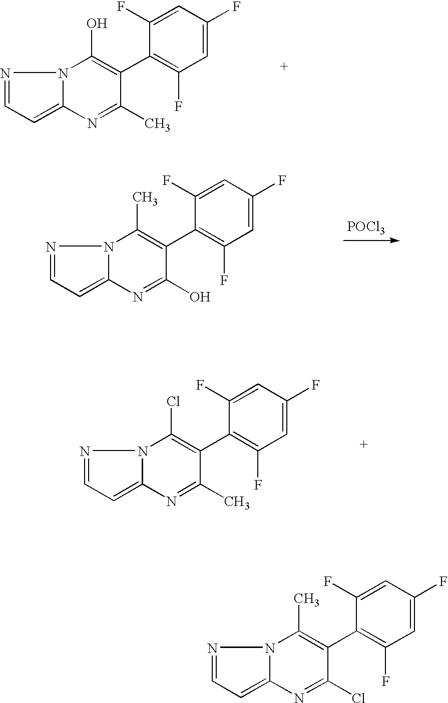

2.8 g (0.01 mol) of the isomer mixture obtained above were heated in 10 ml of phosphorus oxychloride under reflux for 6 hours. After cooling, the reaction mixture was carefully put into ice-water, adjusted to pH 7-8 using sodium carbonate solution and extracted twice with ethyl acetate, and the combined organic phases were dried. Concentration gave 2.6 g of the isomer mixture, which contained about 15% of the title compound.

Chromatography on silica gel (cyclohexane: ethyl acetate mixtures) gave 400 mg of an isomer mixture which contained about 80% of the title compound.

### Step c: 5-Methyl-7-(4-methylpiperidin-1-yl)-6-(2,4,6-trifluorophenyl)pyrazolo[1,5-a]pyrimidine

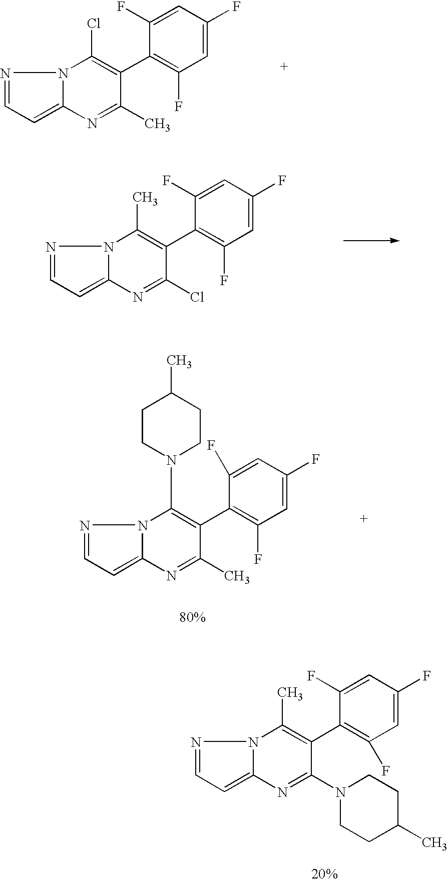

0.4 g of the isomer mixture obtained above (0.0013 mol) and 0.25 g (0.0025 mol) of 4-methylpiperidine were stirred at 40° C. for 4 hours and at room temperature for 12 hours. The reaction mixture was taken up in ethyl acetate, washed twice with 5% strength hydrochloric acid and then with water, dried and concentrated. The residue was chromatographed, which gave 0.176 g of an isomer mixture, which contained about 80% of the title compound.

TABLE I
Intermediates of the formula IV

| | |
|---|---|
| | IV |
| 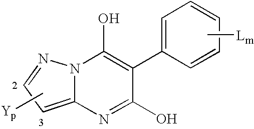 | |

| No. | Yₚ | Lₘ | Phys. data (m.p. [° C.]) |
|---|---|---|---|
| I-1 | 2-CH₃ | 2,4,6-F₃ | 304 |
| I-2 | 2,3-(CH₃)₂ | 2,4,6-F₃ | 375 (decomp.) |
| I-3 | 3-CN | 2,4,6-F₃ | 241 |

TABLE II
Intermediates of the formula V

| | |
|---|---|
| | V |
| 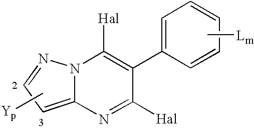 | |

| No. | Yₚ | Hal | Lₘ | Phys. data (m.p. [° C.]) |
|---|---|---|---|---|
| II-1 | 3-Cl | Cl | 2,4,6-F₃ | 128 |
| II-2 | 2-CH₃-3-Cl | Cl | 2,4,6-F₃ | 194 |
| II-3 | 2-CH₃ | Cl | 2,4,6-F₃ | oil |
| II-4 | 2,3 CH₃ | Cl | 2,4,6-F₃ | oil |

αTABLE III
Compounds of the formula I

| | |
|---|---|
| | I |
| 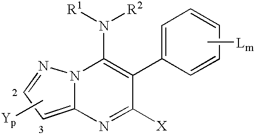 | |

| | | | | | | Phys. data (m.p. |
|---|---|---|---|---|---|---|
| No. | Yₚ | R¹ | R² | X | Lₘ | [° C.]) |
| III-1 | 3-Cl | —(CH₂)₂CH(CH₃)(CH₂)₂— | | Cl | 2,4,6-F₃ | 138 |
| III-2 | 2-CH₃-3-Cl | —(CH₂)₂CH(CH₃)(CH₂)₂— | | Cl | 2,4,6-F₃ | 135 |
| III-3 | 2-CH₃ | —(CH₂)₂CH(CH₃)(CH₂)₂— | | Cl | 2,4,6-F₃ | 185 |
| III-4 | 2-CH₃ | —CH(CH₃)(CH₂)₃— | | Cl | 2,4,6-F₃ | 163 |
| III-5 | 2-CH₃-3-Cl | —CH(CH₃)(CH₂)₃— | | Cl | 2,4,6-F₃ | 152 |
| III-6 | 3-Cl | —CH(CH₃)(CH₂)₃— | | Cl | 2,4,6-F₃ | 69 |
| | | | | | | (decomp.) |
| III-7 | 2,3-CH₃ | —(CH₂)₂CH(CH₃)(CH₂)₂— | | Cl | 2,4,6-F₃ | oil |
| III-8 | 3-CN | —(CH₂)₂CH(CH₃)(CH₂)₂— | | Cl | 2,4,6-F₃ | 8.08* |
| III-9 | 3-CN | (CH₃)₂CHCH(CH₃)— | H | Cl | 2,4,6-F₃ | 8.23* |
| Isomer A | | | | | | |
| III-10 | 3-CN | (CH₃)₂CHCH(CH₃)— | H | Cl | 2,4,6-F₃ | 8.24* |
| Isomer B | | | | | | |
| III-11 | 3-CN | (CH₃)₃CH(CH₃)— | H | Cl | 2,4,6-F₃ | 8.24* |
| Isomer A | | | | | | |
| III-12 | 3-CN | (CH₃)₃CH(CH₃)— | H | Cl | 2,4,6-F₃ | 8.23* |
| Isomer B | | | | | | |
| | | | | | | phys. Daten (Fp. |
| No. | Yₚ | R¹ | R² | X | Lₘ | [° C.]) |
| III-13 | 3-CN | CH₃CH₂— | CH₃CH₂— | Cl | 2,4,6-F₃ | 8.31* |
| III-14 | 3-CN | CH₂C(CH₃)CH₂— | CH₃CH₂ | Cl | 2,4,6-F₃ | 8.35* |
| III-15 | 3-CN | (CH₃)₂CH— | H | Cl | 2,4,6-F₃ | 146 |
| III-16 | 3-CN | CH₃CH₂CH(CH₃)— | H | Cl | 2,4,6-F₃ | 119 |

| | | | | | |
|---|---|---|---|---|---|
| III-17 | 3-CH₃ | —(CH₂)₂CH(CH₃)(CH₂)₂— | Cl | 2,4,6-F₃ | 153 |
| III-18 | 3-CH₃ | —(CH₂)₂CH(CH₃)(CH₂)₂— | OH | 2,4,6-F₃ | 234 |
| III-19 | 3-Cl | —(CH₂)₂CH(CH₃)(CH₂)₂— | CN | 2,4,6-F₃ | 7.9* |
| III-20 | 3-Cl | —(CH₂)₂CH(CH₃)(CH₂)₂— | OCH₃ | 2,4,6-F₃ | 8.13* |
| III-21 | 3-CN | —(CH₂)₂CH(CH₃)(CH₂)₂— | OCH₃ | 2,4,6-F₃ | 8.62* |
| III-22 | 3-CN | —(CH₂)₂CH(CH₃)(CH₂)₂— | CN | 2,4,6-F₃ | 150 |
| III-23 | 3-CN | —(CH₂)₂CH(CH₃)(CH₂)₂— | CH₃ | 2,4,6-F₃ | 8.27* |

*¹H-NMR(CDCl₃): 1H(H-2)

### EXAMPLES OF THE ACTION AGAINST HARMFUL FUNGI

The fungicidal action of the compounds of the formula I was demonstrated by the following experiments:

The active compounds were separately prepared as a stock solution of 0.25% by weight of active compound in acetone or DMSO. 1% by weight of emulsifier Uniperol® EL (wetting agent having emulsifying and dispersing action based on ethoxylated alkylphenols) was added to this solution, and the solution was diluted with water to the desired concentration.

### Use Example 1

### Activity Against Gray Mold on Capsicum Leaves Caused by Botrytis Cinerea, Protective Application

Capsicum seedlings of the cultivar “Neusiedler Ideal Elite” were, after 4-5 leaves had fully developed, sprayed to runoff point with an aqueous suspension having the concentration of active compound stated below. The next day, the treated plants were inoculated with a spore suspension of Botrytis cinerea comprising 1.7×10⁶ spores/ml in a 2% strength aqueous biomalt solution. The test plants were subsequently placed in a climatized chamber at 22 to 24° C. and high atmospheric humidity. After 5 days, the extent of the fungal infection on the leaves could be determined visually in %.

In this test, the with 250 ppm of the active compound III-1 or III-6 of Table III showed an infection of at most 7%, whereas the untreated plants were 90% infected.

### Use Example 2

### Activity Against Mildew on Cucumber Leaves Caused by Sphaerotheca Fuliginea, Protective Application

Leaves of potted cucumber seedlings of the cultivar “Chinese snake” were, at the cotyledon stage, sprayed to runoff point with an aqueous suspension having the concentration of active compound stated below. 20 hours after the spray coating had dried on, the plants were inoculated with an aqueous spore suspension of mildew of cucumber (Sphaerotheca fuliginea). The plants were then cultivated in a greenhouse at temperatures between 20 and 24° C. and 60 to 80% relative atmospheric humidity for 7 days. The extent of the mildew development was then determined visually in % infection of the cotyledon area.

In this test, the with 250 ppm of active compound III-1 or III-6 of Table III showed no infection, whereas the untreated plants were 90% infected.+−

1. A substituted pyrazolopyrimidine of the formula I 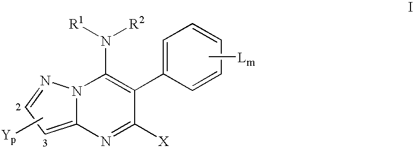
in which the substituents are as defined below:
L independently of one another are halogen, C₁-C₆-alkyl, C₂-C₆-alkenyl, C₁-C₆-haloalkyl, C₁-C₆-alkoxy, amino, NHR, NR₂, cyano, S(═O)ₙA¹ or C(═O)A²,
R is C₁-C₈-alkyl or C₁-C₈-alkylcarbonyl;
A¹ is hydrogen, hydroxyl, C₁-C₈-alkyl, C₁-C₈-alkylamino or di-(C₁-C₈-alkyl)amino;
n is 0, 1 or 2;
A² is C₂-C₈-alkenyl, C₁-C₈-alkoxy, C₁-C₆-haloalkoxy or one of the groups mentioned under A¹;
m is 0, 1, 2, 3, 4 or 5;
R¹ is C₁-C₈-alkyl, C₁-C₈-haloalkyl, C₃-C₆-cycloalkyl, C₃-C₆-halocycloalkyl, C₂-C₈-alkenyl, C₄-C₁₀-alkadienyl, C₂-C₈-haloalkenyl, C₃-C₆-cycloalkenyl, C₂-C₈-alkynyl, C₂-C₈-haloalkynyl or C₃-C₆-cycloalkynyl, phenyl, naphthyl or a five- to ten-membered saturated, partially unsaturated or aromatic heterocycle which contains one to four heteroatoms from the group consisting of O, N and S,
R² is hydrogen or one of the groups mentioned under R¹;
R¹ and R² together with the nitrogen atom to which they are attached may also form a five- or six-membered ring which may be interrupted by an atom from the group consisting of O, N and S and/or which may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain;
where R¹ and/or R² may be substituted by one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, or a five- to ten-membered saturated, partially unsaturated or aromatic heterocycle which contains one to four heteroatoms from the group consisting of O, N and S,
where these aliphatic, alicyclic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups mentioned in these radicals contain 2 to 8 carbon atoms;
and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;
X is halogen, cyano, OH, C₁-C₄-alkyl, C₁-C₄-alkoxy or C₁-C₂-haloalkoxy;
Y is a five- to ten-membered saturated, partially unsaturated or aromatic heterocycle which contains one to four heteratoms from the group consisting of O, N and S, or one of the groups mentioned under X (which groups may be substituted by one to four identical or different groups R^{a}), nitro, amino, —CHO, —NHCO—NH—C₁-C₆-alkyl, —NHCO—O—C₁-C₆-alkyl, —CO—NH₂;
p is 1 or 2, where the groups Y may be different if p=2;
p is O if the group X is cyano, C₁-C₄-alkyl, C₁-C₄-alkoxy or C₁-C₄-haloalkoxy.

2. The compound of the formula I according to claim 1 in which X is halogen, CN, OH, C₁-C₄-alkyl or C₁-C₄-alkoxy.

3. The compound of the formula I according to claim 1 in which X is halogen, CN or C₁-C₄-alkyl.

4. The compound of the formula I according to claim 1 in which X is halogen or C₁-C₄-alkyl.

5. The compound of the formula I according to claim 1 in which X is halogen.

6. The compound of the formula I according to any of claims 1 to 5 in which R¹ and R² are as defined below:
R¹ is C₁-C₆-alkyl, C₁-C₈-haloalkyl, C₃-C₆-cycloalkyl, C₃-C₆-halocycloalkyl, C₂-C₈-alkenyl, C₂-C₈-haloalkenyl, C₂-C₈-alkynyl; and
R² is hydrogen or C₁-C₄-alkyl; or
R¹ and R² together with the nitrogen atom to which they are attached may also form a five- or six-membered saturated or unsaturated ring which may carry one or two substituents from the group consisting of halogen, C₁-C₆-alkyl and C₁-C₆-haloalkyl.

7. The compound of the formula I according to claim 1 in which the phenyl group substituted by Lₘ is the group C 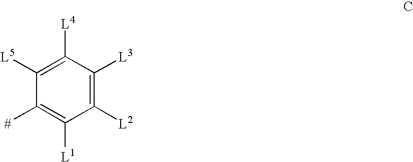
in which # is the point of attachment with the triazolopyrimidine skeleton and
L¹ is fluorine, chlorine, CH₃ or CF₃;
L²,L⁴ independently of one another are hydrogen or fluorine;
L³ is hydrogen, fluorine, chlorine, cyano, CH₃, OCH₃ or COOCH₃; and
L⁵ is hydrogen, fluorine or CH₃.

8. The process for preparing the compound of the formula I according to claim 1 in which X is halogen by reacting substituted 3-aminopyrazole derivatives of the formula II in which Yₚ is as defined for formula I according to claim 1 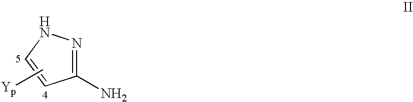
and 2-phenylmalonates III 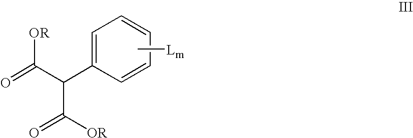
to give dihydroxypyrazolopyrimidines IV, 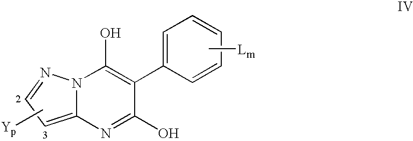
halogenating IV to give halogenpyrazolopyrimidines V 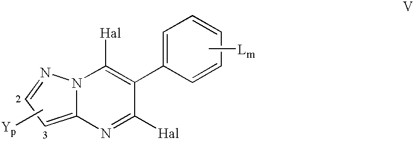
and reacting V with amines of the formula VI.

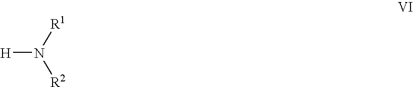

9. The process for preparing the compounds of the formula I according to claim 1 in which the groups X and Y are halogen and Y is located in the 3-position of the pyrazolopyrimidine skeleton by halogenating compounds of the formula VII 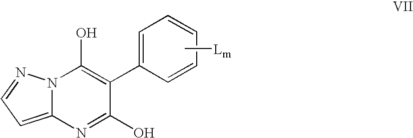
in which the variables are as defined for formula I and Hal is a halogen atom, to give trihalopyrazolopyrimidines of the formula VIII 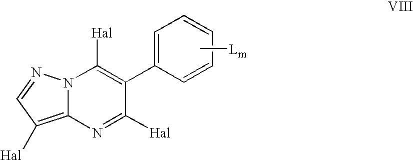
and reacting VIII with amines of the formula VI according to claim 5 to give compounds of the formula I.A.

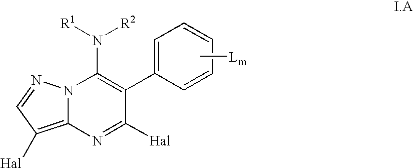

10. The process for preparing the compounds of the formula I according to claim 1 in which X is cyano, C₁-C₆-alkoxy or C₁-C₂-haloalkoxy, by reacting halopyrazolopyrimidines of the formula I in which X is halogen with compounds of the formula IX
M-X′ IX
in which X′ is cyano, C₁-C₆-alkoxy or C₁-C₂-haloalkoxy and M is an ammonium, tetraalkylammonium or alkali metal or alkaline earth metal cation, to give compounds of the formula I.B.

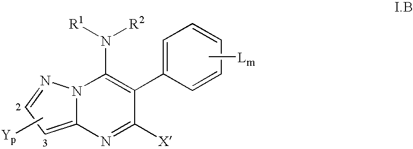

11. The process for preparing the compounds of the formula I according to claim 1 in which X is C₁-C₄-alkyl, by reacting halopyrazolopyrimidines of the formula I in which X is halogen with compounds of the formula X
M^{y}(X″)_{y} X
in which X″ is a C₁-C₄-alkyl group and M is a metal ion of the valency Y, in particular B, Zn or Sn, under transition metal catalysis in an inert solvent or diluent, to give compounds of the formula I.C 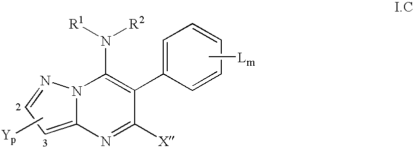
in which X″ is C₁-C₄-alkyl.

12. The process for preparing the compounds of the formula I according to claim 1 in which X is C₁-C₄-alkyl, by reacting substituted 3-aminopyrazoles of the formula II according to claim 5 with ketoesters of the formula XI 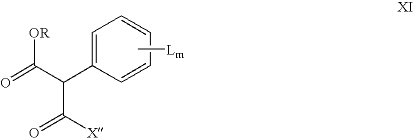
in which R is a C₁-C₄-alkyl group and X″ is C₁-C₄-alkyl, to give hydroxypyrazolopyrimidines of the formula XII 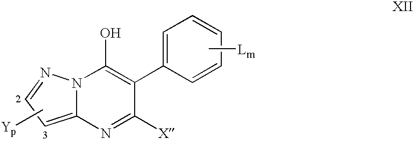
and halogenating XII to give compounds of the formula XIII 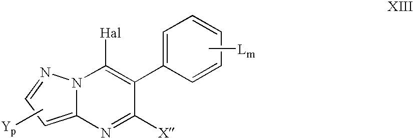
and reacting XIII with amines of the formula VI according to claim 5 to give compounds of the formula I in which X is C₁-C₄-alkyl.

13. The process for preparing compounds of the formula I according to claim 1 in which X is CN or C₁-C₄-alkoxy, by partially hydrolyzing the 5,7-dihalopyrazolopyrimidines of the formula V according to claim 5 with aqueous sodium hydroxide solution to give 5-halo-7-hydroxypyrazolopyrimidines Va 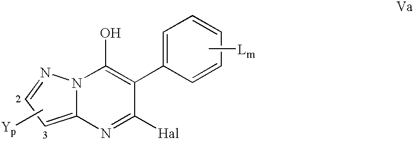
reacting Va with organometallic compounds of the formula IX according to claim 10 to give the compounds of the formula XIIa 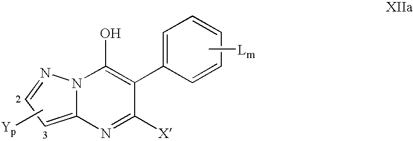
in which X′ is CN or C₁-C₄-alkoxy and M is as defined in claim 10; followed by halogenation of XIIa to give compounds of the formula XIIIa 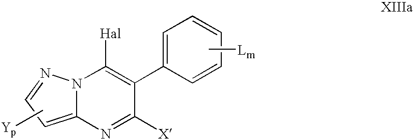
and reaction of the compounds of the formula XIIIa with amines of the formula VI according to claim 8 to give compounds of the formula I.D.

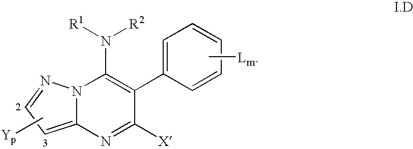

14. An intermediate of the formula IV, V, Va, VIII, XII, XIIa, XIII or XIIIa according to any one of claims 8, 9, 12 or 13.

15. A composition suitable for controlling harmful fungi, which composition comprises a solid or liquid carrier and a compound of the formula I according to claim 1.

16. A process for controlling phytopathogenic harmful fungi, which process comprises treating the fungi or the materials, plants, the soil or seeds to be protected against fungal attack with an effective amount of a compound of the formula I according to claim 1.
